(19) United States
(12) Patent Application Publication
(10) Pub. No.: US 2022/0153718 A1
(43) Pub. Date: May 19, 2022

(54) SYNTHESIS OF STABLE AMORPHOUS APALUTAMIDE

(71) Applicant: OLON S.P.A., Rodano (MI) (IT)

(72) Inventors: Valentina Grande, Rodano (MI) (IT); Barbara Novo, Rodano (MI) (IT); Mara SADA, Rodano (MI) (IT); Gabriele Ferretti, Rodano (MI) (IT); Alessia Manfredi, Rodano (MI) (IT)

(73) Assignee: OLON S.P.A., Rodano (MI) (IT)

(21) Appl. No.: 17/438,948

(22) PCT Filed: Mar. 9, 2020
(86) PCT No.: PCT/IB2020/052011

§ 371 Date: Sep. 14, 2021

(30) Foreign Application Priority Data
Mar. 15, 2019 (IT) 102019000003839

(51) Int. Cl. C07D 401/04

(52) U.S. Cl. CPC C07D 401/04; C07B 2200/13

(57) ABSTRACT

The present invention relates to a process for the preparation of apalutamide in stable amorphous form. The invention also relates to a novel intermediate crystalline form, called form X, which gives rise to said amorphous form, and a process for obtaining said form X.

### FIELD OF INVENTION

The present invention relates to a process for the preparation of apalutamide in stable amorphous form.

Apalutamide is the international non-proprietary name of the compound 4-{7-[6-cyano-5-(trifluoromethyl)pyridin-3-yl]-8-oxo-6-sulphanylidene-5,7-diazaspiro[3,4]octan-5-yl}-2-fluoro-N-methylbenzamide having the formula (I).

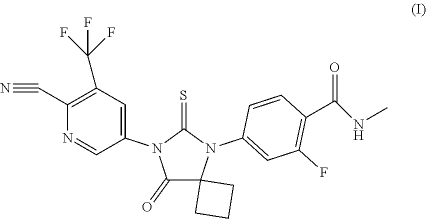 [SMILES: CNC(=O)c1ccc(N2C(=S)N(c3cnc(C#N)c(C(F)(F)F)c3)C(=O)C23CCC3)cc1F]

Apalutamide is the active ingredient present in the medicament Erleada® approved by the FDA in the USA and by the EMA in Europe for the treatment of non-metastatic castration-resistant prostate cancer.

Apalutamide, classified in the Biopharmaceutics Classification System as a class 2 medicament, requires high doses (240 mg a day divided into four administrations).

The purpose of the invention is to obtain a highly bioavailable form of apalutamide in order to reduce the daily dose of active ingredient, thus making the treatment more effective and reducing its side effects.

One method of making an active ingredient more bioavailable is to formulate the API in amorphous form. The amorphous form of an active ingredient is known to be much more soluble and much more bioavailable than its various crystalline forms.

It is also known that the amorphous form is less stable in some cases, and partly converts to the more stable crystalline form. It is therefore necessary to synthesise a stable amorphous form of apalutamide (i.e. containing no trace of the crystalline form) for use in the formulation of a medicament.

### PRIOR ART

Apalutamide is disclosed in U.S. Pat. No. 8,445,507, filed by Aragon Pharmaceuticals.

In view of its structural complexity, apalutamide has various crystalline forms, including the amorphous form.

WO2013184681, filed by Aragon Pharmaceuticals, describes forms A, B, C, D, E, F, G, I and J, and the preparation of capsules containing a pure crystalline form. WO2016124149 describes form I and form II. WO2018112001 describes forms T1 to T19 and claims forms T2, T6, T11 and T13.

Some of said crystalline forms are solvated and have a high solvent content, while others are poorly stable or obtainable by a process that is not industrially scalable.

The amorphous form of apalutamide is described in WO2019016747 as a solid amorphous dispersion with various excipients such as copovidone, polyvinyl acetate phthalate, cellulose acetate phthalate or hydroxypropyl methylcellulose phthalate (HPMC), or as a co-amorphous form with L-tartaric acid, citric acid, saccharin and sucralose.

To obtain the amorphous solid dispersion or the co-amorphous form, apalutamide in amorphous form is never isolated; a solution of apalutamide is amorphised by known techniques with the appropriate excipient or the appropriate molecule with which it is to be coformulated, dissolved in a suitable solvent. These processes indicate that the amorphous form obtained is poorly stable, so in order to obtain a stable amorphous form it must be prepared from a solution containing excipients, or coformulated.

WO2019016747 also describes a process for the preparation of amorphous apalutamide but not in great detail, and without providing the stability data of the amorphous apalutamide obtained. The process described and claimed involves dissolving apalutamide in a suitable solvent selected from acetone and ethanol or mixtures thereof and isolation of the amorphous form of apalutamide by a technique selected from known amorphisation techniques, such as evaporation of solvent at atmospheric pressure or alternatively at low pressure using a rotary evaporator, spray drying, freeze drying, filtration of the amorphous product or the thin-film drying process.

In particular, the process described, which involves dissolving apalutamide in acetone, ethanol or mixtures thereof and isolating amorphous apalutamide by filtering the product from the mixture of solvents used, differs from patent application WO2013184681, which describes the use of said solvents or mixtures thereof to obtain form A. The Applicant, when repeating the same experiment in the laboratory, obtained form A, not the amorphous form (Example no. 3).

A process for the preparation of an amorphous form of apalutamide which is stable in the absence of excipients, or other molecules that function as stabilisers, is not known.

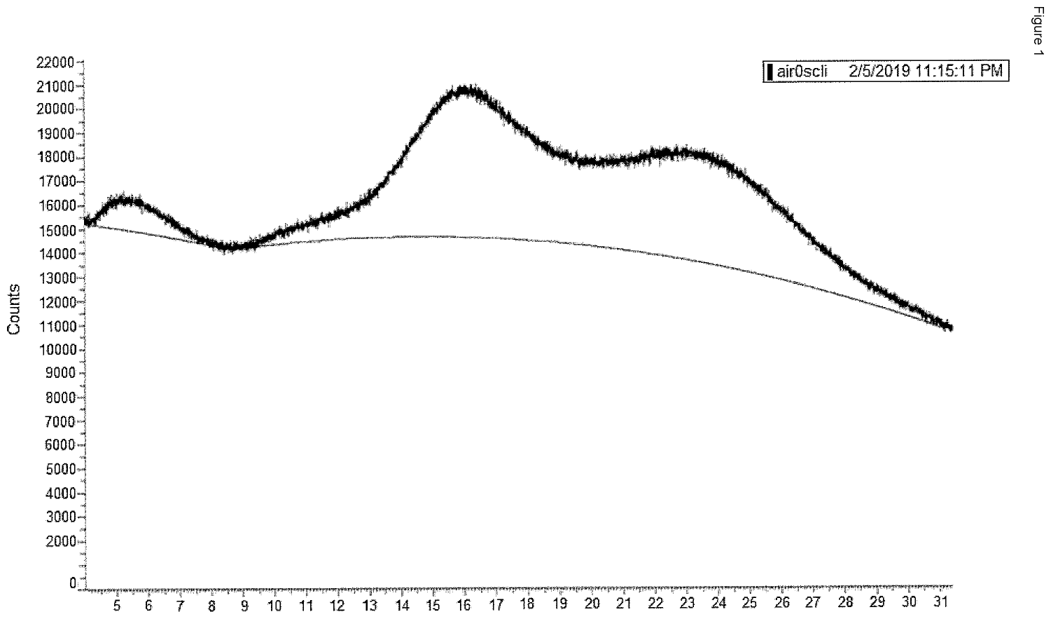

FIG. 1: XRPD pattern of amorphous apalutamide.

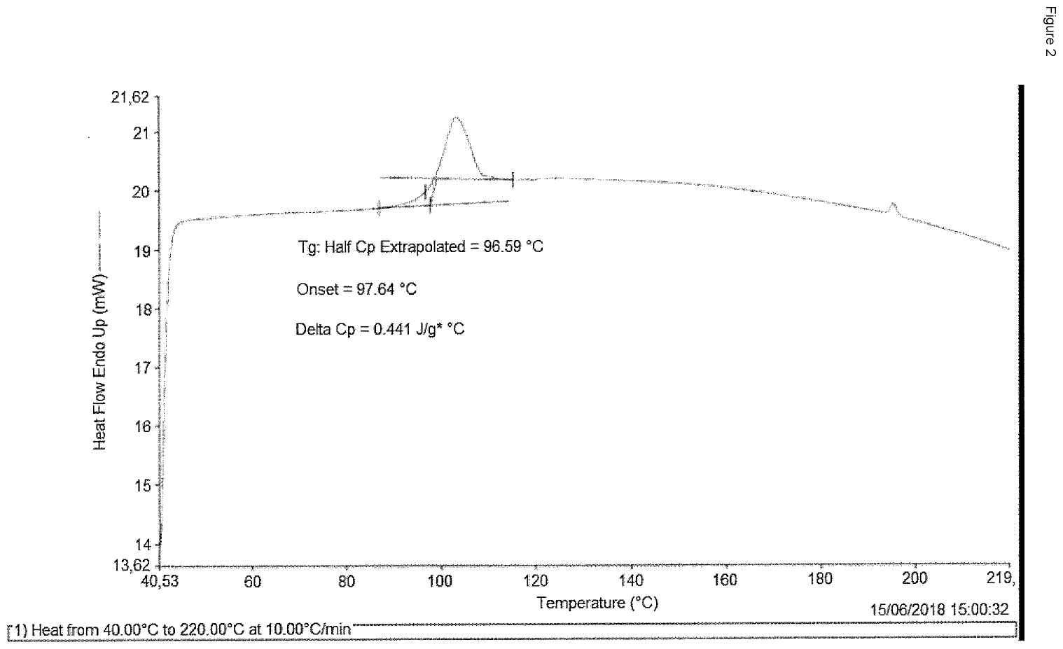

FIG. 2: DSC curve of apalutamide, amorphous form.

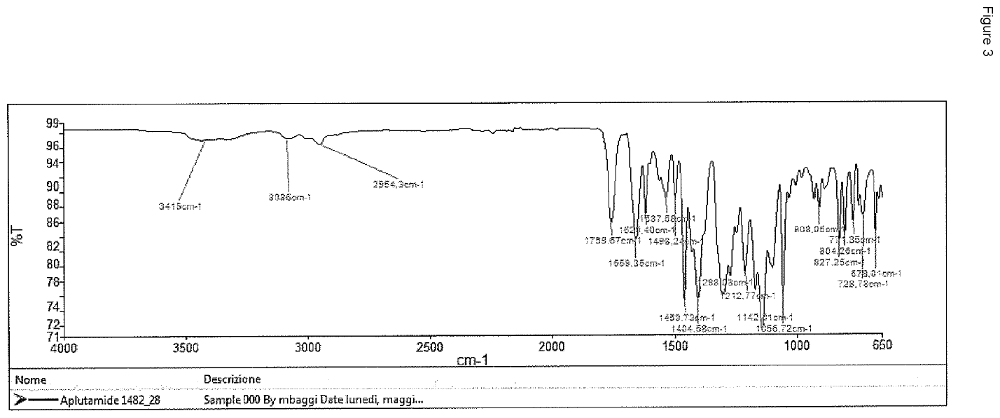

FIG. 3: IR spectrum of apalutamide, amorphous form.

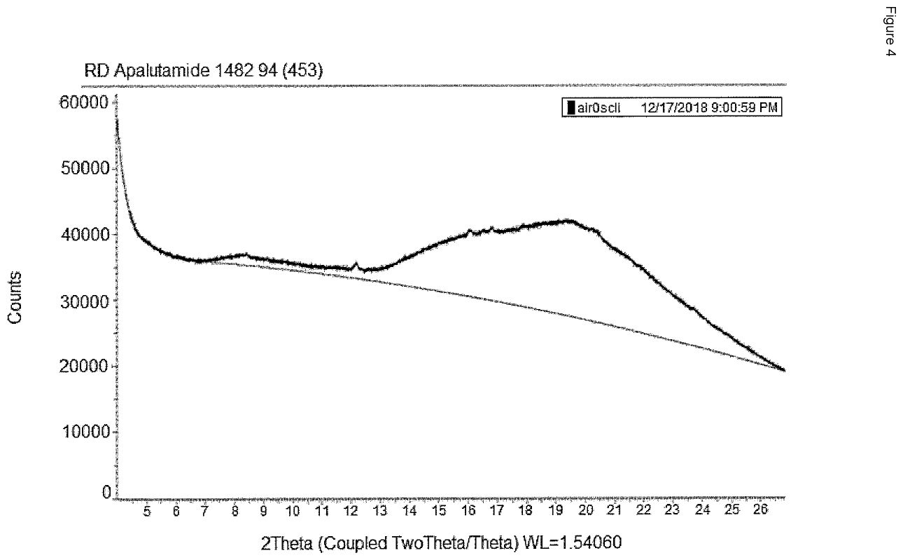

FIG. 4: XRPD spectrum of unstable amorphous apalutamide obtained from form B.

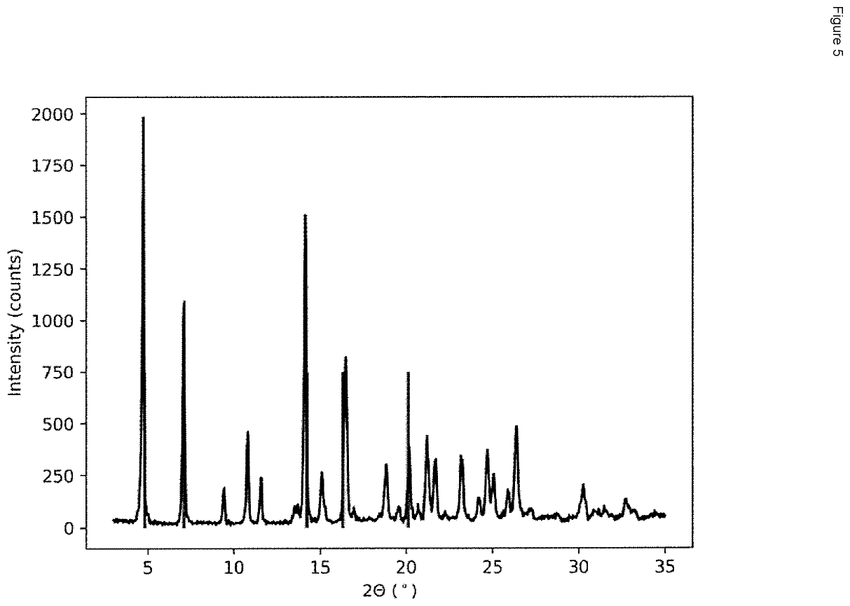

FIG. 5: XRPD spectrum of apalutamide, form A.

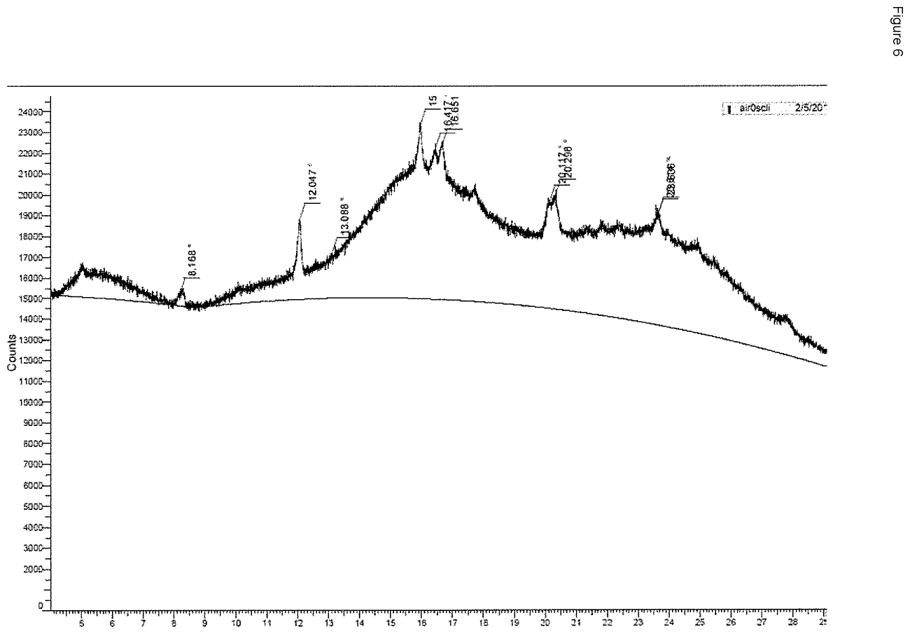

FIG. 6: XRPD spectrum of mixture of amorphous form, form A and form B of apalutamide obtained by forced-air drying.

### DESCRIPTION OF THE INVENTION

The invention relates to a process for the preparation of apalutamide of formula (I) in stable amorphous form which comprises:
a) dissolving apalutamide in a polar aprotic solvent;
b) filtering the resulting solution;
c) heating the solution to 50-80° C.;
d) adding an anti-solvent;
e) maintaining the mixture at 50-80° C. for 10-60 minutes;
f) cooling the mixture to 0-20° C.;
g) filtering the crystalline apalutamide;
h) drying the crystalline apalutamide at 50-110° C. for a time ranging between 4 h and 120 h.

The crude apalutamide is dissolved in step a) in a polar aprotic solvent, preferably acetonitrile. The solution is then filtered (step b) to eliminate the presence at this step of any precipitate and/or foreign body, and heated (step c) to a temperature ranging between 50 and 80° C., preferably between 55 and 70° C.

An anti-solvent (step d), preferably water, is added to the resulting solution, and the temperature of the mixture is maintained at 50-80° C. for 10-60 minutes, preferably at 55-70° C., for 15-30 minutes.

When the anti-solvent is water, an acetonitrile/water ratio ranging between 2/1 and 1/2 is used, the volumes of acetonitrile and water preferably being in the ratio of 1.4/1.

The mixture (step e) is cooled to 0-20° C., preferably to 0-5° C., and the resulting crystalline apalutamide is filtered.

The crystalline apalutamide is dried at a temperature ranging between 50 and 110° C. for a time ranging between 4 h and 120 h, preferably at 80-100° C., for a time ranging between 12 and 90 h, preferably at 90° C. for 18 h, in the presence or absence of humidity, to produce the stable amorphous form.

The stable amorphous form of apalutamide obtained by the process described above presents an XRPD spectrum as shown in FIG. 1, demonstrating that it contains no trace of crystalline form (Bruker D8 advance diffractometer 2.5°-40° 2θ. Scan step 0.02° and 0.5 s per step, Cuα).

The FTIR spectrum and DSC curve of the stable amorphous form of apalutamide are shown in FIGS. 2 and 3.

The stable amorphous form of apalutamide obtained by the process claimed proved stable, even after forced-air drying.

Forced-air drying here means drying at 90° C. for over 100 hours, under vacuum, in a stove.

The amorphous form obtained by evaporation of the solvent after dissolution of form B exhibits traces of form B after lengthy acquisition of the XRPD spectrum (FIG. 4, same conditions as FIG. 1) and when subjected to forced-air drying under the same conditions as described above, is converted to a mixture of amorphous form, form A and form B, as shown in FIG. 6 (same conditions as in FIG. 1).

The invention is described in greater detail in the examples below.

### Example 1: Synthesis of Apalutamide in Stable Amorphous Form

Apalutamide is solubilised in 5 volumes of acetonitrile, and the solution is heated to 45° C. and filtered. 3.6 volumes of demineralised water are added, and the mixture is heated to 60-65° C. The mixture is left under stirring for 15 minutes. The mixture is cooled to 0-5° C. The product is filtered through a Büchner funnel and washed with a water/acetonitrile mixture.

The product recovered is dried at 45° C. under vacuum for at least 6 h, to obtain apalutamide in crystalline form.

The product recovered as apalutamide in crystalline form, directly wet or partly dried, when subjected to further drying under vacuum at 90° C. for at least 18 h, produces amorphous apalutamide, as shown in FIG. 1.

### Example 2: Test for Recovery of Amorphous Apalutamide from Form B

Apalutamide form B is dissolved in acetonitrile and filtered. The solution is dried under vacuum at the temperature of 60° C., and the solid is then recovered.

An amorphous product with clear presence of form B is obtained, as will be seen from the XRPDs shown in FIG. 4.

### Example 3: Recovery of Apalutamide Form A from Ethanol

Amorphous apalutamide is suspended in 20 volumes of ethanol at room temperature and left under stirring for 48 h. The product is recovered by filtration and dried at 40° C. under vacuum for at least 12 h.

Apalutamide form A is isolated, as shown in FIG. 5.

1. A process for the preparation of the stable amorphous form of apalutamide 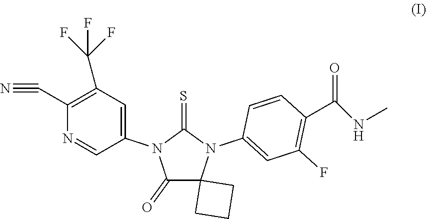 [SMILES: CNC(=O)c1ccc(N2C(=S)N(c3cnc(C#N)c(C(F)(F)F)c3)C(=O)C23CCC3)cc1F]
comprising the sequence of the following steps:
a) dissolving apalutamide in a polar aprotic solvent;
b) filtering the resulting solution;
c) heating the solution to 50-80° C.;
d) adding an antisolvent;
e) keeping the mixture at 50-80° C. for 10-60 minutes;
f) cooling the mixture to 0-20° C.;
g) filtering the crystalline apalutamide;
h) drying the crystalline apalutamide at 50-110° C. for a time ranging from 4 h to 120 h.

2. Process according to claim 1 wherein in step a) the polar aprotic solvent is acetonitrile.

3. Process according to claim 1 wherein step c) is performed at 55-70° C.

4. Process according to claim 1 wherein the antisolvent used in step d) is water.

5. Process according to claim 4 wherein the acetonitrile to water ratio ranges from 2/1 to 1/2.

6. Process according to claim 1 wherein the temperature in step e) ranges from 55 to 70° C. and is maintained for 15-30 minutes.

7. Process according to claim 1 wherein the temperature in step f) is maintained between 0 and 5° C.

8. Process according to claim 1 wherein step h) is performed at 80-100° C. for a time ranging from 12 to 90 h.

9. Process according to claim 5 wherein acetonitrile and water are in a 1.4/1 ratio.

10. Process according to claim 8, wherein step h) is performed 90° C. for 18 h.
